(19) United States
(12) Patent Application Publication
(10) Pub. No.: US 2025/0329441 A1
(43) Pub. Date: Oct. 23, 2025

(54) HEALTHCARE MANAGEMENT SYSTEM

(71) Applicant: MyMedVita Inc., Wayzata, MN (US)

(72) Inventors: Dane Stimart, Minneapolis, MN (US); Stephen Parente, Wayzata, MN (US); Carrie Parente, Wayzata, MN (US)

(21) Appl. No.: 19/025,685

(22) Filed: Jan. 16, 2025

Related U.S. Application Data
(60) Provisional application No. 63/621,487, filed on Jan. 16, 2024.

(51) Int. Cl. G16H 40/20; G16H 10/60

(52) U.S. Cl. CPC G16H 40/20; G16H 10/60

(57) ABSTRACT

A healthcare shopping and management system is disclosed herein which assists users in searching for medical providers based on insurer-negotiated pricing, directly scheduling appointments, making payments, aggregating personal health records, and utilizing analytics to suggest preventative health screenings. The system architecture includes interfaces for searching, scheduling, paying, health records access, geolocation of providers, cost ratings, and an analytics engine leveraging clinical guidelines. Database schema and data models as well as example user interfaces are also disclosed.

### CLAIM OF PRIORITY

This application claims the benefit of priority under 35 U.S.C. § 119 (e) to U.S. Provisional Patent Application Ser. No. 63/621,487, filed on Jan. 16, 2024, which is incorporated by reference herein in its entirety.

### BACKGROUND

Navigating healthcare pricing, insurance coverage, scheduling appointments, and tracking personal health history can be complex and frustrating for patients. The lack of transparency in medical pricing makes it extremely difficult for consumers to get a fair price and understand what they will be expected to pay. Prices can vary wildly between different hospitals and medical providers for the same services, but this information is rarely made clear upfront. This prevents consumers from being able to effectively comparison shop. The opacity in pricing also leads to certain groups like the uninsured being charged exorbitant amounts.

Overall, the confusing and obscure nature of medical pricing is detrimental to both the economy and individuals. It inflates healthcare costs significantly and leaves patients struggling with unexpected medical bills. More pricing transparency and consumer protections would assist to remedy this issue that impacts so many people's finances and well-being.

In addition, under the current healthcare system, patients face numerous barriers to accessing their own medical records. Requesting records often involves filling out lengthy forms, waiting weeks or longer for a response, and potentially paying fees. This lack of transparency and impediments to accessing one's health history is problematic on many levels. If obtaining medical records was a simpler, faster process, it would greatly empower patients. Having easy access to their full records allows individuals to make more informed choices related to their care, get second opinions, and have continuity if seeing multiple providers. Open access reduces confusion and helps patients be actively involved stewards of their health. Enabling people to readily view their medical information is also likely to build greater trust in patient-provider relationships as well as the healthcare system overall. Removing the obstacles people currently face to see their own health documentation would thus provide tangible individual benefits while also improving systemic transparency shortcomings.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 provides an overview of the components of a healthcare management system. It shows the user inputs, system processing, and outputs displayed.

FIG. 2A gives a high level overview of the software functionality of Marketplace capabilities of the present technology.

FIG. 2B gives a high level overview of the software functionality of Clinic Companion capabilities of the present technology.

FIG. 2C illustrates an example cloud architecture with client devices connecting to backend system processing components deployed in the cloud.

FIG. 2D gives a high level overview of the software functionality of Healthy Pathways capabilities of the present technology.

FIG. 2E illustrates an aspect of the subject matter in accordance with one embodiment.

FIG. 3 illustrates an example cloud architecture with client devices connecting to backend system processing components deployed in the cloud.

FIG. 4A gives program flows of the software functionality and flow of Marketplace capabilities of the present technology.

FIG. 4B gives program flows of the software functionality and flow of Clinic Companion capabilities of the present technology.

FIG. 4C gives program flows of the software functionality and flow or Health Encounters capabilities of the present technology.

FIG. 4D gives program flows of the software functionality and flow of Healthy Pathways capabilities of the present technology.

FIGS. 5A-5C illustrate the data model with entities and attributes for the system database schema showing example relationships between entities.

FIG. 6A illustrates an example app in accordance with an embodiment.

FIG. 6B illustrates an example app in accordance with an embodiment.

FIG. 6C illustrates an example app in accordance with an embodiment.

FIG. 6D illustrates an example app in accordance with an embodiment.

FIG. 6E illustrates an example app in accordance with an embodiment.

FIG. 6F illustrates an example app in accordance with an embodiment.

FIG. 6G illustrates an example app in accordance with an embodiment.

FIG. 6H illustrates an example app in accordance with an embodiment.

FIG. 6I illustrates an example app in accordance with an embodiment.

FIG. 6J illustrates an example app in accordance with an embodiment.

FIG. 6K illustrates an example app in accordance with an embodiment.

FIG. 6L illustrates an example app in accordance with an embodiment.

FIG. 6M illustrates an example app in accordance with an embodiment.

FIG. 6N illustrates an example app in accordance with an embodiment.

FIG. 6O illustrates an example app in accordance with an embodiment.

FIG. 6P illustrates an example app in accordance with an embodiment.

FIG. 6Q illustrates an example app in accordance with an embodiment.

FIG. 6R illustrates an example app in accordance with an embodiment.

FIG. 6S illustrates an example app in accordance with an embodiment.

FIG. 7A illustrates an example app in accordance with an embodiment.

FIG. 7B illustrates an example app in accordance with an embodiment.

FIG. 7C illustrates an example app in accordance with an embodiment.

FIG. 7D illustrates an example app in accordance with an embodiment.

FIG. 7E illustrates an example app in accordance with an embodiment.

FIG. 7F illustrates an example app in accordance with an embodiment.

FIG. 7G illustrates an example app in accordance with an embodiment.

FIG. 7H illustrates an example app in accordance with an embodiment.

FIG. 7I illustrates an example app in accordance with an embodiment.

FIG. 7J illustrates an example app in accordance with an embodiment.

FIG. 7K illustrates an example app in accordance with an embodiment.

FIG. 7L illustrates an example app in accordance with an embodiment.

FIG. 7M illustrates an example app in accordance with an embodiment.

FIG. 7N illustrates an example app in accordance with an embodiment.

FIG. 7O illustrates an example app in accordance with an embodiment.

FIG. 7P illustrates an example app in accordance with an embodiment.

FIG. 7Q illustrates an example app in accordance with an embodiment.

FIG. 7R illustrates an example app in accordance with an embodiment.

FIG. 7S illustrates an example app in accordance with an embodiment.

FIG. 7T illustrates an example app in accordance with an embodiment.

FIG. 8 illustrates an aspect of the subject matter in accordance with one embodiment.

FIG. 9 illustrates an aspect of the subject matter in accordance with one embodiment.

### DETAILED DESCRIPTION

The following detailed description illustrates various non-limiting example embodiments for a healthcare management system. One skilled in the art will understand other variants and modifications possible within the scope and spirit of the disclosure.

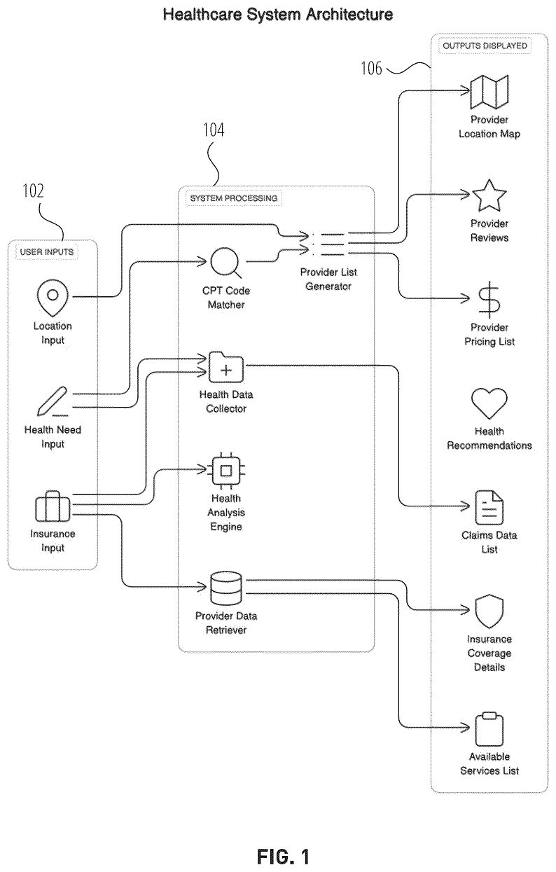

Referring now to FIG. 1 there is illustrated an overview of the components of a healthcare management system according to one example embodiment.

The User Inputs components 102 allow users to provide location (“user-specified location”), health needs, insurance details, and interact with the healthcare system architecture. The Location Input allows users to input their location. The Health Need Input allows users to describe health needs and conditions. The Insurance Input allows users to provide insurance details.

The System Processing components 104 perform key functions. The CPT Code Matcher matches services to standard billing codes. The Provider List Generator produces a list of providers based on location and services. The Health Data Collector gathers user health data. The Health Analysis Engine analyzes the collected data. The Provider Data Retriever obtains provider information.

The Outputs Displayed components 106 present results and recommendations. The Provider Location Map shows provider locations on a map. The Provider Reviews shows reviews. The Provider Pricing List displays pricing. The Health Recommendations gives service suggestions based on analysis. The Claims Data List presents insurance claims information. The Insurance Coverage Details outlines user coverage. The Available Services List displays accessible services.

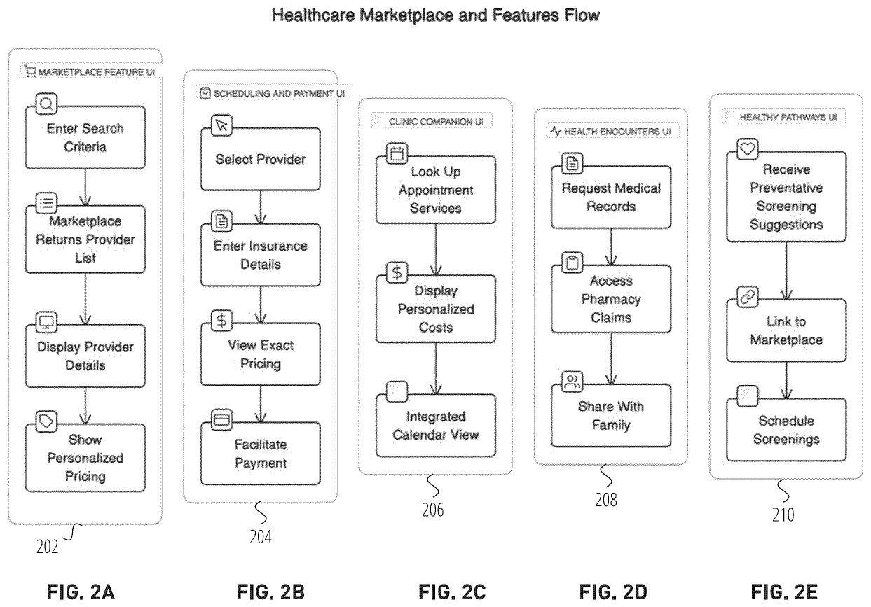

Referring now to FIGS. 2A, 2B, 2C, 2D there is illustrated a high level overview of the software functionality provided by one example embodiment.

As shown in FIG. 2A a Marketplace Feature flow 202 allows users to input search criteria. This criteria is passed to a provider database to return a list of matching providers. Details for each provider are then formatted and displayed, along with personalized pricing generated based on the user's insurance and other factors.

As shown in FIG. 2B, once a provider is selected, a Scheduling and Payment flow 204 takes over. Insurance information input by the user is validated and used to generate exact pricing details for the selected provider and services. Payment can then be facilitated through this module by obtaining the user's payment information and providing to an online payment system.

As shown in FIG. 2C a Clinic Companion module 206 maintains data on upcoming and past appointments. Users can view appointment services and personalized costs in an integrated calendar view.

In addition to facilitating in-person appointments, the healthcare management system incorporates robust telemedicine capabilities. Users can not only find and book appointments but also conduct virtual consultations directly through the app. The integrated video conferencing feature is HIPAA-compliant and includes tools for secure file sharing and e-prescriptions. This telemedicine functionality expands access to healthcare services, particularly for users in remote areas or those with mobility limitations, and provides a convenient option for follow-up appointments and minor health concerns.

As shown in FIG. 2D Health Encounters flow 208 enables automated requests for medical records from provider EHR systems. Pharmacy claims data is also accessed through APIs and formatted for user access and sharing.

Finally, as shown in FIG. 2E Healthy Pathways flow 210 uses patient screening guidelines to generate preventative screening suggestions which link back to the Marketplace Feature to schedule screenings.

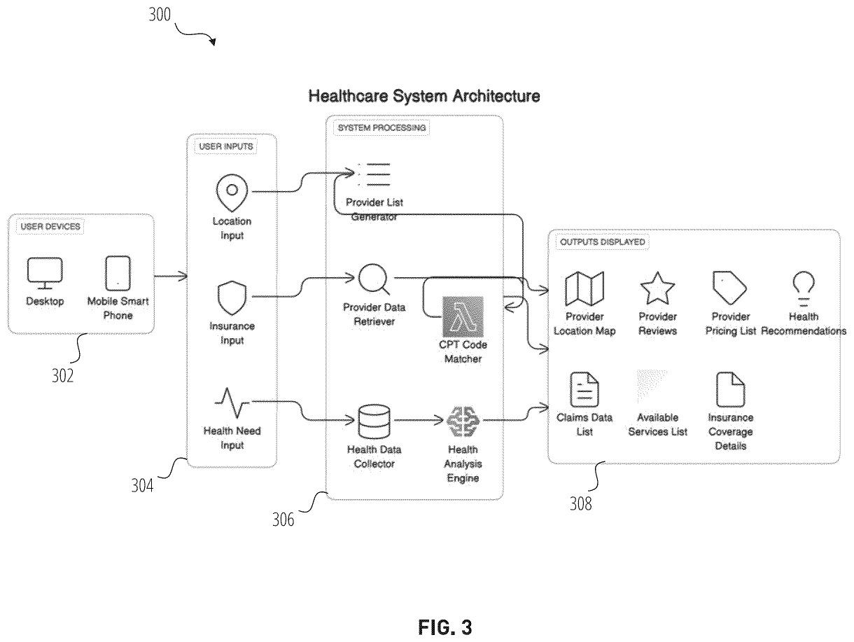

FIG. 3 illustrates an example embodiment of a cloud architecture 300 according to an example embodiment. Client devices 302 such as desktop computers and mobile smart phones allow users to input data such as their location, insurance information, and health needs. These user inputs 304 are communicated over local and/or wide area networks to backend system processing components that generate relevant outputs.

The system processing components 306, all or a portion of which may be deployed in the cloud, include a provider list generator, provider data retriever, CPT code matcher, health data collector, and health analysis engine. These components process the user input and collect relevant health data and provider information.

The outputs 308 generated from the system processing are then displayed back to the user client devices over the networks. These outputs include a provider location map, provider reviews, claims data, list of available services, health pricing information, insurance coverage details, and health recommendations. Accordingly, in one example embodiment a cloud architecture connects user devices to backend healthcare system processing to collect inputs, analyze health data, and display relevant outputs back to the users.

According to one embodiment the healthcare management system prioritizes data security and privacy, implementing robust measures to protect sensitive user information. All data transmissions are encrypted using industry-standard protocols, and the system is fully compliant with healthcare regulations such as HIPAA. Users have granular control over their data, including the ability to specify what information is shared and with whom. Regular security audits and penetration testing ensure the ongoing integrity of the system, providing users with peace of mind as they manage their healthcare information.

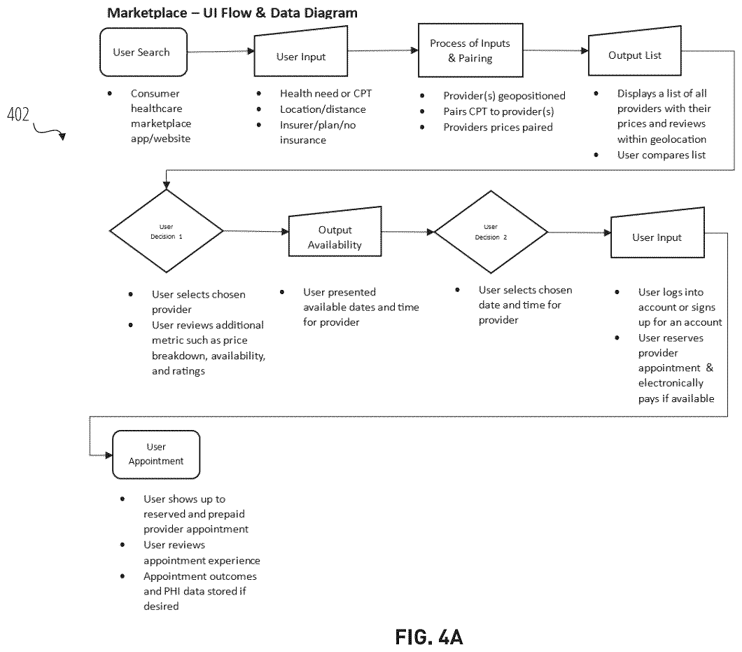

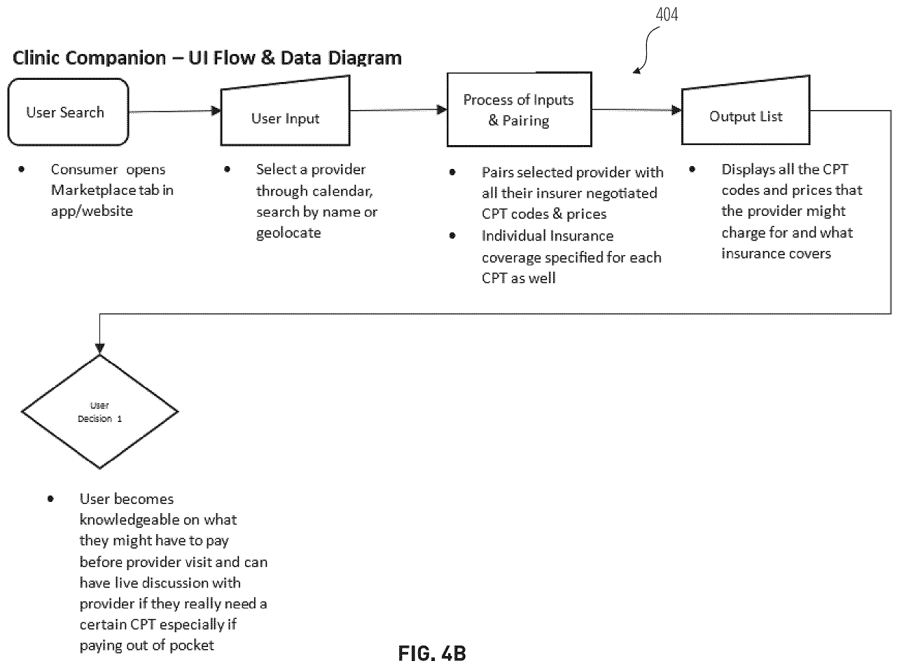

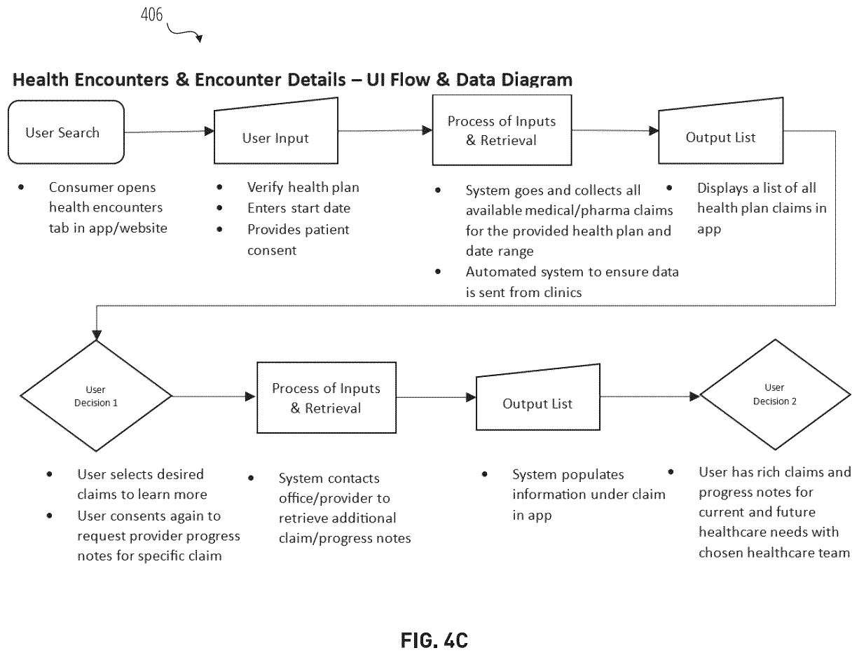

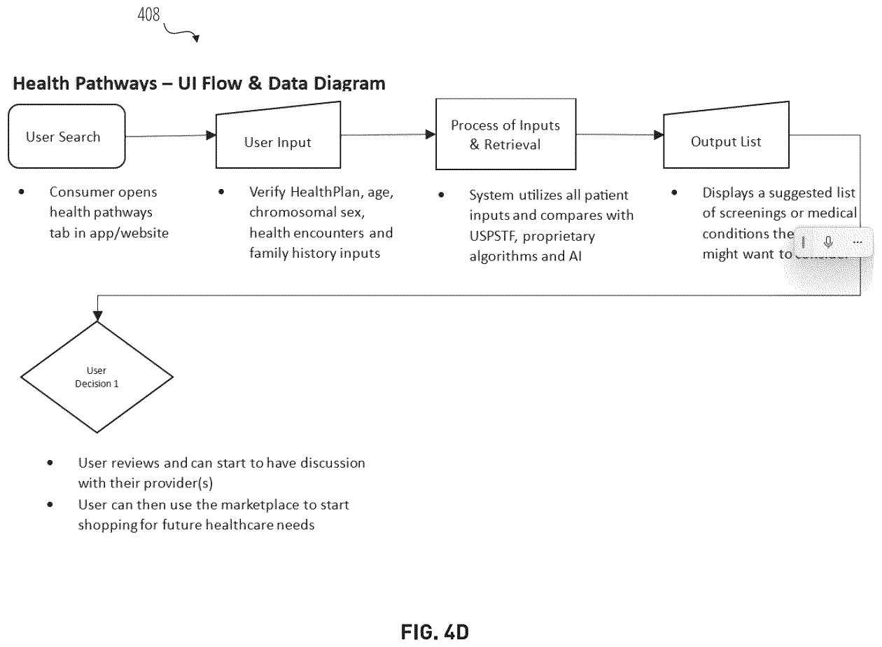

Referring now to FIGS. 4A, 4B, 4C and 4D there are illustrated data and program flows according to an example embodiment.

A marketplace flow 402 in FIG. 4A provides a User Search capability allowing User Input Details to be input. The user inputs their specific health need or medical procedure code (CPT code) and their location and desired search distance/radius. Also, the user specifies their health insurance details or notes they have no insurance.

The system matches the user's specified CPT code(s) to providers who offer those medical services, and the system generates a list of providers matching the CPT services within the user's location radius, showing the price each provider charges for those services as well as customer reviews for each provider.

The user is shown the generated Output List of matching providers with the pricing info and reviews for each one, with exact geopositioning/location shown on a map.

FIG. 4B illustrates a Clinic Companion function set 404 wherein a user provides User Input Details to find a provider either by date availability shown on a calendar, searching for them by name, or using geopositioning to select one on a map.

The system takes the specific provider selected by the user and retrieves all medical procedure codes (CPT codes) they offer along with the pre-negotiated pricing their office has established with each insurer (also referred to herein as “insurer-negotiated prices” or “insurer-negotiated pricing”.

The user is shown the full list of medical services (CPT codes) offered by the chosen provider in an Output List, including the exact price they charge for each service and what amount is covered by the user's specified insurance plan.

FIG. 4C illustrates the Health Encounters capabilities 406 according to an embodiment. The user provides Input Details and consents to allow their personal health data to be collected/accessed, and provides details of their health insurance plan which will be verified. The user also specifies the beginning date of the desired date range of health records to be collected.

Leveraging the user-verified health plan details and specified date range, the system goes and gathers all available medical claims and pharmaceutical claims data.

To ensure the highest level of data integrity and security, in one alternate embodiment, the healthcare management system leverages blockchain technology. Each health record, transaction, and data exchange is recorded on a distributed ledger, creating an immutable audit trail. This blockchain implementation not only enhances security but also facilitates more efficient and trustworthy data sharing between healthcare providers, insurers, and patients. The use of smart contracts on the blockchain can automate and streamline processes such as insurance claims and payment distributions, reducing administrative overhead and potential errors.

The healthcare management system is designed with interoperability at its core, seamlessly integrating with existing healthcare IT infrastructure. It interfaces with Electronic Health Records (EHRs) and Health Information Exchanges (HIEs), allowing for real-time data exchange and updates. This integration ensures that users have access to their complete health information, regardless of where it originated, and enables healthcare providers to have a comprehensive view of a patient's health status. The system's interoperability also facilitates smoother transitions of care and reduces the likelihood of duplicate tests or procedures.

Expanding beyond traditional healthcare data sources, the system integrates with a wide range of mobile health (mHealth) devices and applications. Users can connect their wearable fitness trackers, smart scales, blood pressure monitors, and other IoT health devices to the platform. This real-time data integration enhances the accuracy of health recommendations and enables proactive health management. The system can alert users to potential health issues based on trends in their mHealth data, encouraging early intervention and promoting overall wellness.

The user is presented with a complete Output List of all their health insurance claims falling within the selected date range.

Illustrated in FIG. 4D is a Health Pathways capability 408 enabling the user to provide User Input for example confirmed health plan info, their age, biological sex, personal health visit data, and family medical history.

The system feeds all the user inputs into proprietary health analysis algorithms and AI models, comparing the data against clinical standards like the U.S. Preventative Services Task Force (USPSTF) guidelines.

Based on the thorough analysis, the system generates Output Details including a customized list of recommended health screenings or possible medical conditions for the user to discuss with their doctor for their specific situation and risk factors.

Recognizing the significant impact of social and environmental factors on health outcomes, the system incorporates social determinants of health into its analysis and recommendations. Users can input information about their living conditions, education level, employment status, and other relevant factors. The system then uses this data to provide more holistic health recommendations, including suggestions for community resources, educational programs, and lifestyle modifications tailored to the user's specific circumstances. This comprehensive approach addresses health from a broader perspective, potentially leading to improved long-term outcomes.

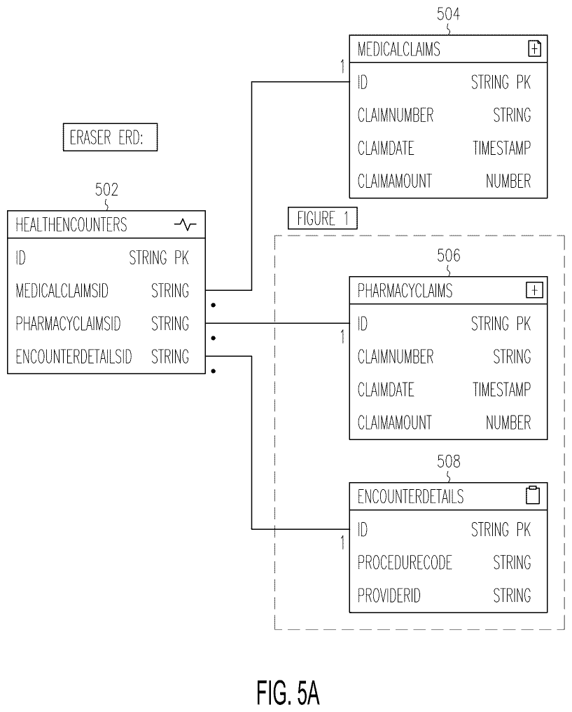

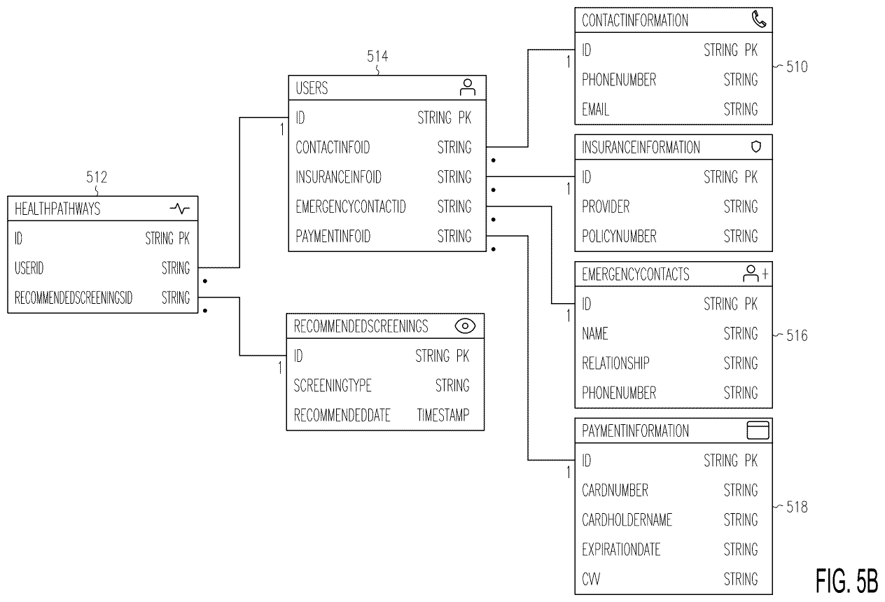

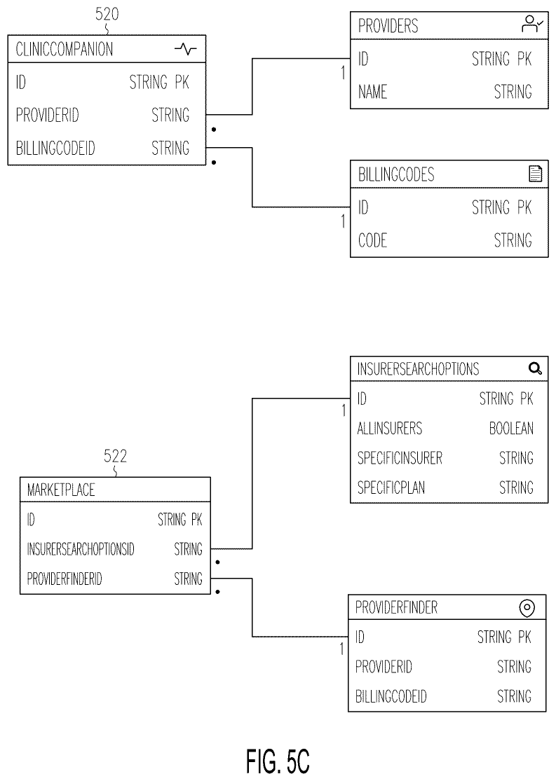

FIG. 5 illustrates a data model for a healthcare service shopping and management system. The entities are connected to model a healthcare service shopping and management system database schema. The foreign key relationships allow data to be accessed across multiple related tables.

There are 11 entities illustrated:
healthEncounters 502
medicalClaims 504
pharmacyClaims 506
encounterDetails 508
contactInformation 510
healthPathways 512
users 514
emergencyContacts 516
paymentInformation 518
clinicCompanion 520
marketplace 522

The healthEncounters entity has 4 attributes:
id (primary key)
medicalClaimsId (foreign key to medicalClaims entity)
pharmacyClaimsId (foreign key to pharmacyClaims entity)
encounterDetailsId (foreign key to encounterDetails entity)

The medicalClaims entity has 4 attributes:
id (primary key)
claimNumber
claimDate
claimAmount

The pharmacyClaims entity has 4 attributes:
id (primary key)
claimNumber
claimDate
claimAmount

The encounterDetails entity has 3 attributes:
id (primary key)
procedureCode
providerId

The contactInformation entity has 3 attributes:
id (primary key)
phoneNumber
email

The healthPathways entity has 5 attributes:
id (primary key)
userld (foreign key to users entity)
contactInfold (foreign key to contactInformation entity)
insurancelnfold
emergencyContactld (foreign key to emergencyContacts entity)

The users entity has 3 attributes:
id (primary key)
recommendedScreeningsId (foreign key to recommendedScreenings entity)
paymentInfold (foreign key to paymentInformation entity)

The emergencyContacts entity has 4 attributes:
id (primary key)
name
relationship
phoneNumber

The paymentInformation entity has 3 attributes:
id (primary key)
cardNumber
cardHolderName

The clinicCompanion entity has 2 attributes:
id (primary key)
providerld

The marketplace entity has 3 attributes:
id (primary key)
insurerSearchOptionsId (foreign key to insurerSearchOptions entity)
providerFinderld (foreign key to providerFinder entity)

There are also 4 additional entities not directly connected to the main diagram:

The recommendedScreenings entity has 3 attributes:
id (primary key)
screeningType
recommendedDate

The provider entity has 2 attributes:
id (primary key)
name

The billingCodes entity has 3 attributes:
id (primary key)
code
expirationDate

The insurerSearchOptions entity has 3 attributes:
id (primary key)
allInsurers
specificInsurer
specificPlan

The providerFinder entity has 2 attributes:
id (primary key)
providerld
billingCodeld

The healthEncounters entity relates to the medicalClaims, pharmacyClaims, and encounterDetails entities via foreign key relationships.

The healthPathways entity relates to the users, contactInformation, emergencyContacts entities via foreign key relationships.

The users entity relates to the recommendedScreenings and paymentInformation entities via foreign key relationships.

The marketplace entity relates to the insurerSearchOptions and providerFinder entities via foreign key relationships.

Beyond its primary function as a healthcare management tool for individuals, the system has the potential to contribute significantly to medical research and public health initiatives. With user consent, anonymized data from the system can be aggregated and analyzed to identify health trends, assess the effectiveness of treatments, and inform public health policies. This research capability transforms individual health management into a collective effort to improve healthcare outcomes on a broader scale. The system's comprehensive data set, combining clinical, lifestyle, and social determinants of health, offers unique insights that could drive advancements in personalized medicine and population health management.

The providerFinder entity relates to the provider and billingCodes entities via foreign key relationships.

To summarize:
healthEncounters is related to medicalClaims, pharmacyClaims, encounterDetails
healthPathways is related to users, contactInformation, emergencyContacts
users is related to recommendedScreenings, paymentInformation
marketplace is related to insurerSearchOptions, providerFinder
providerFinder is related to provider, billingCodes

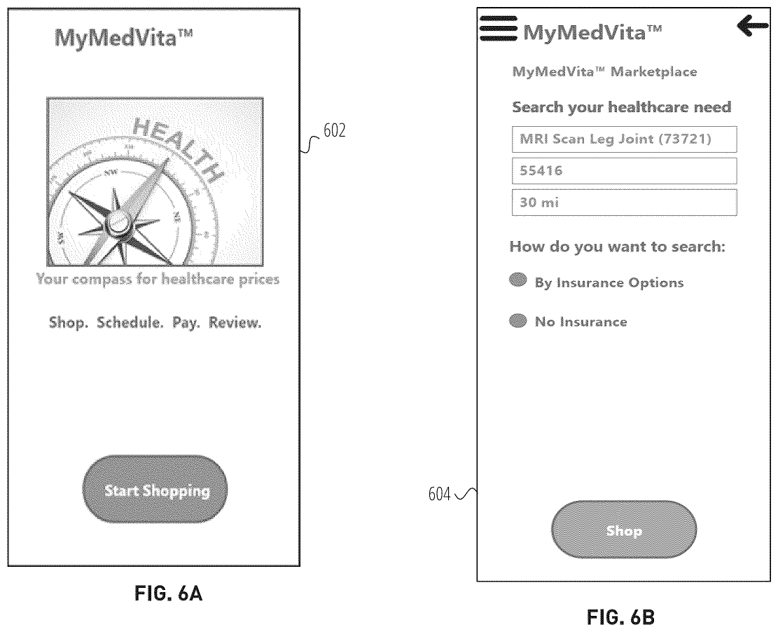

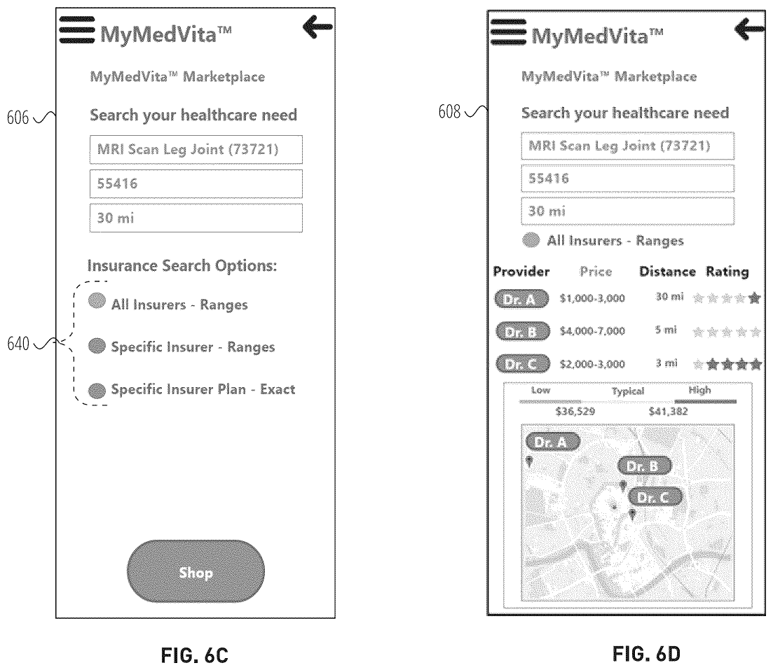

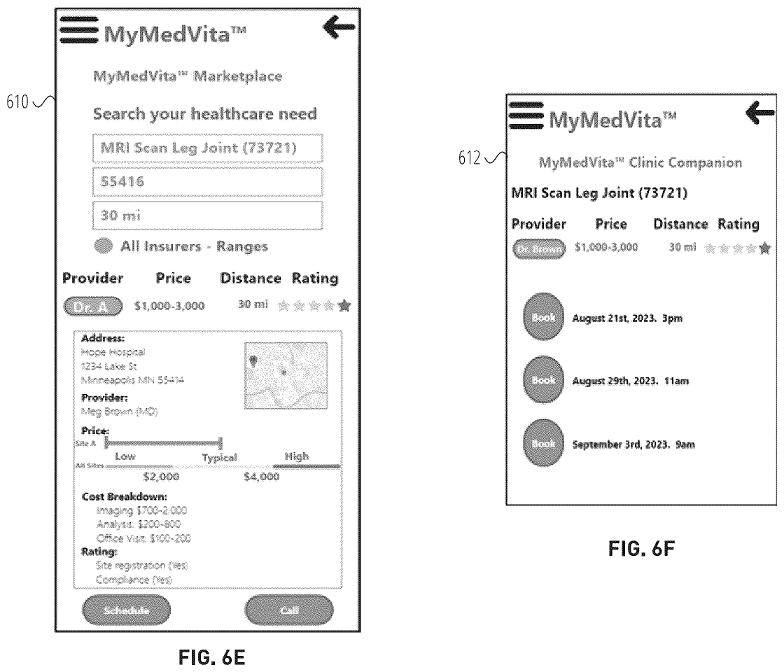

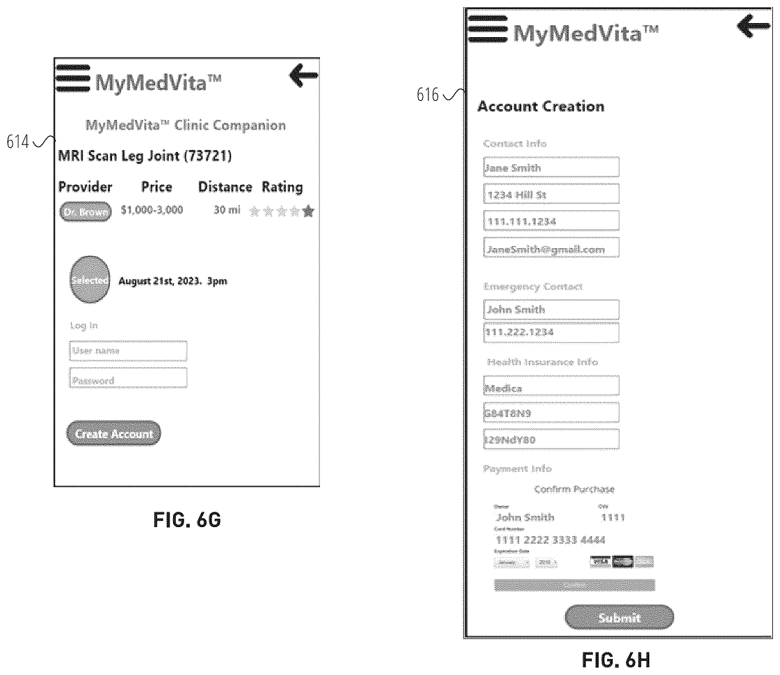

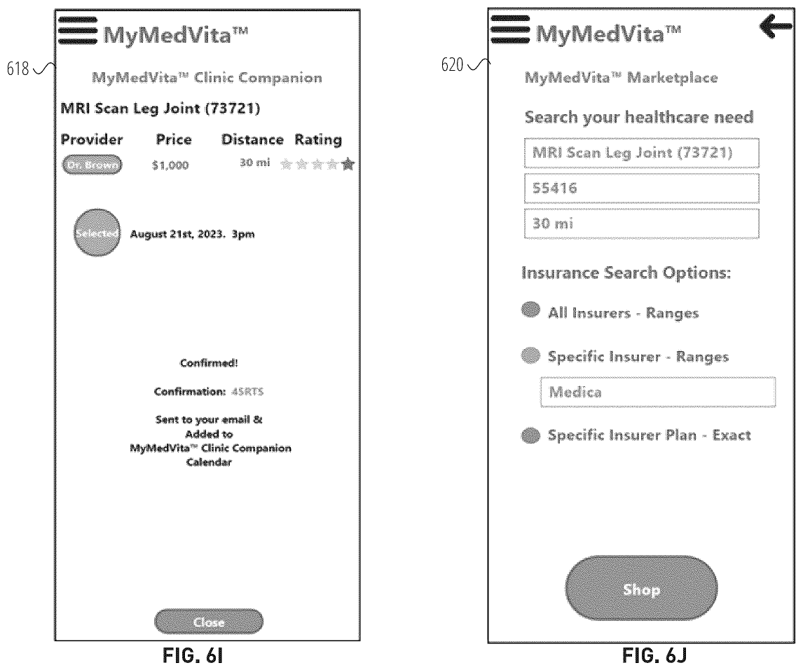

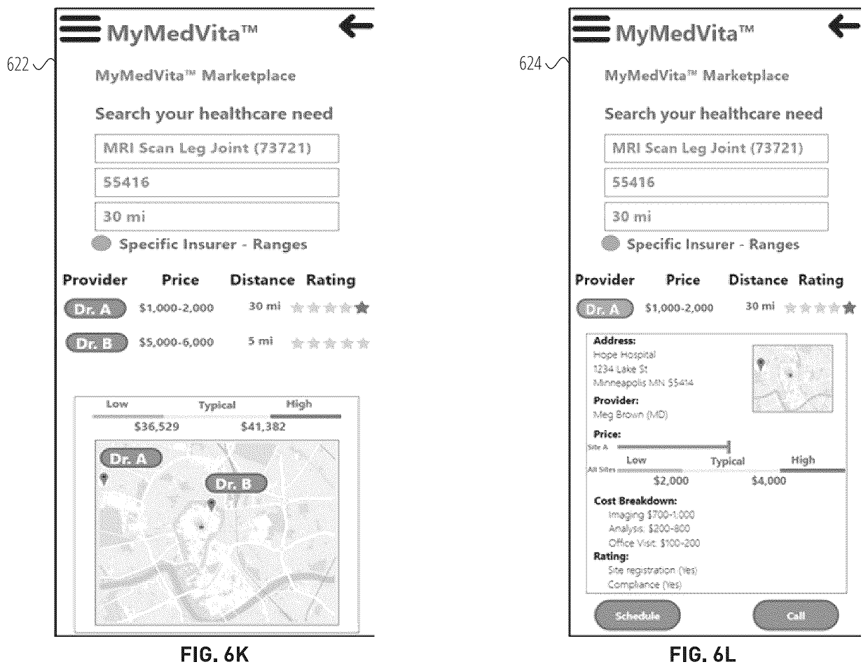

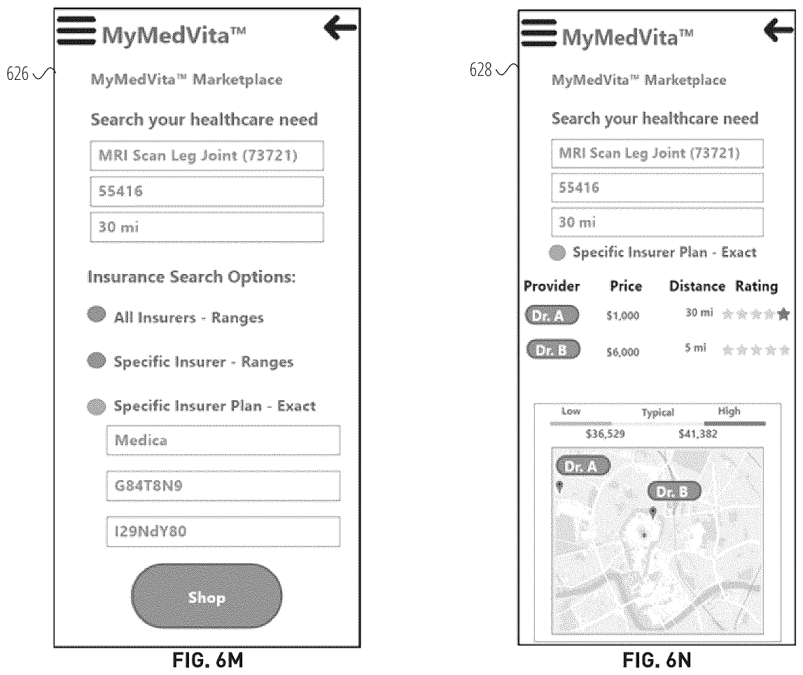

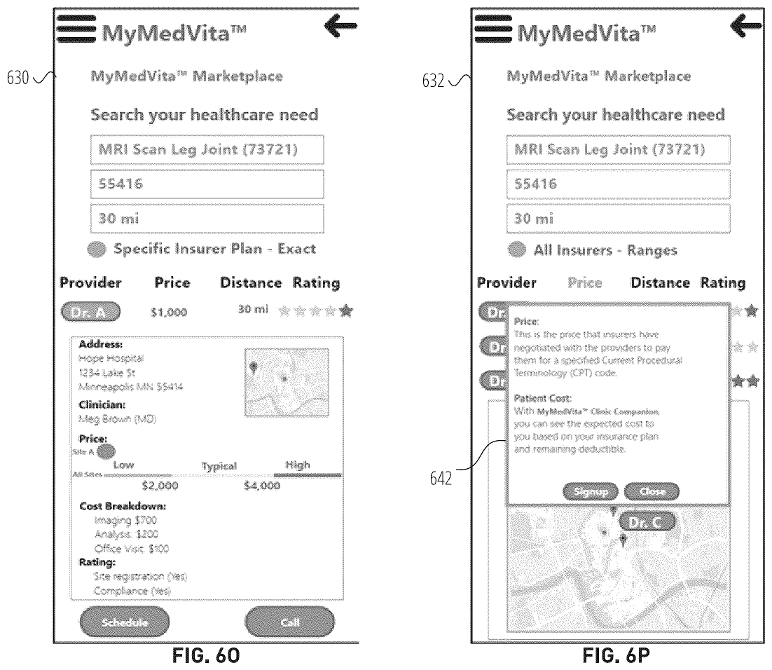

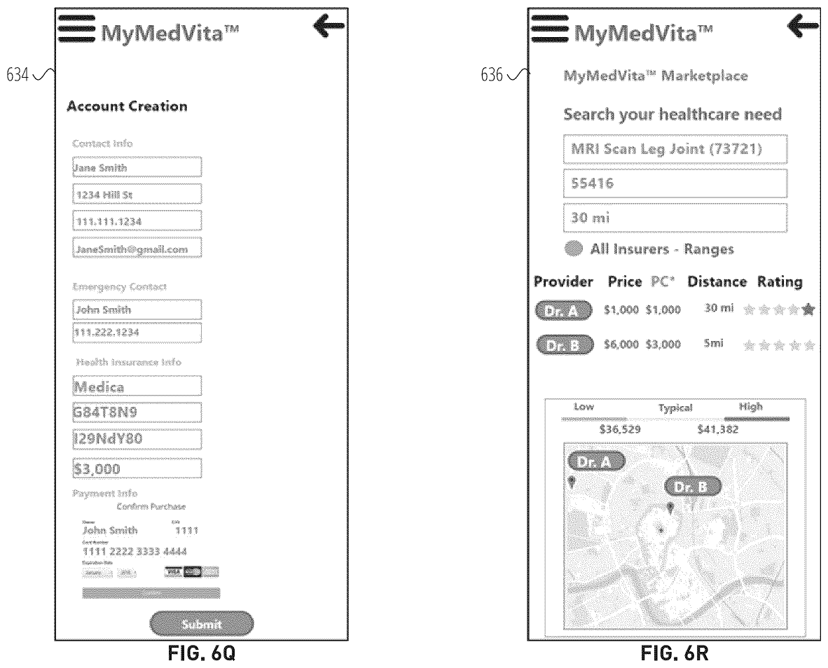

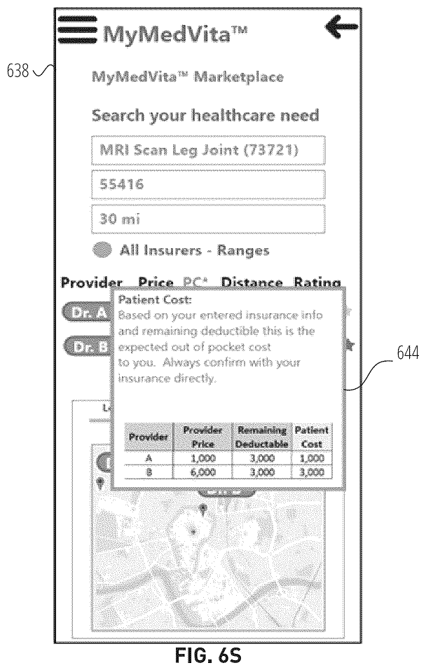

As shown in FIG. 6A, the app home page 602 showing logo, compass graphic, and text stating ‘Your compass for healthcare prices’. Additional text lists abilities to shop, schedule, pay, and review. A ‘Start Shopping’ button is shown.

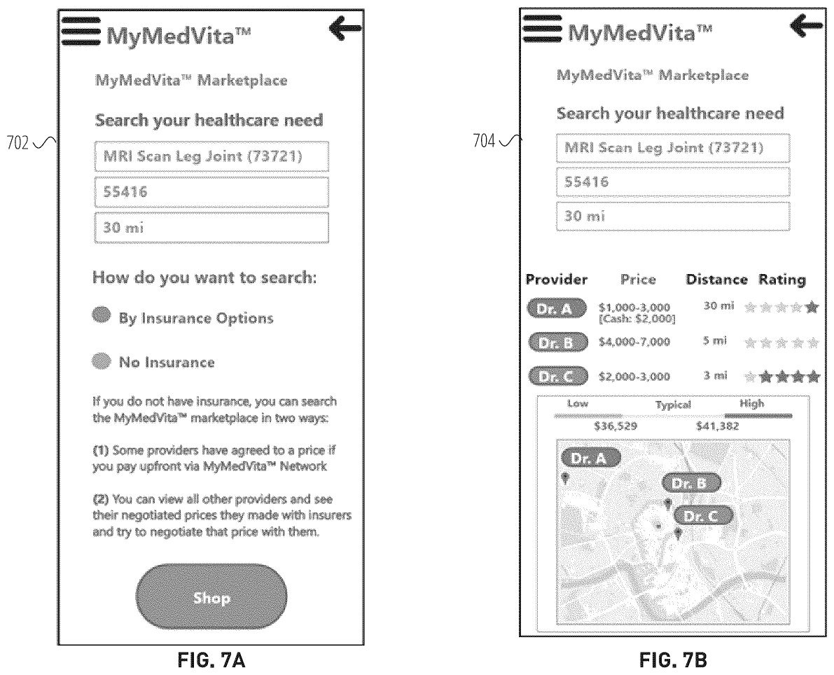

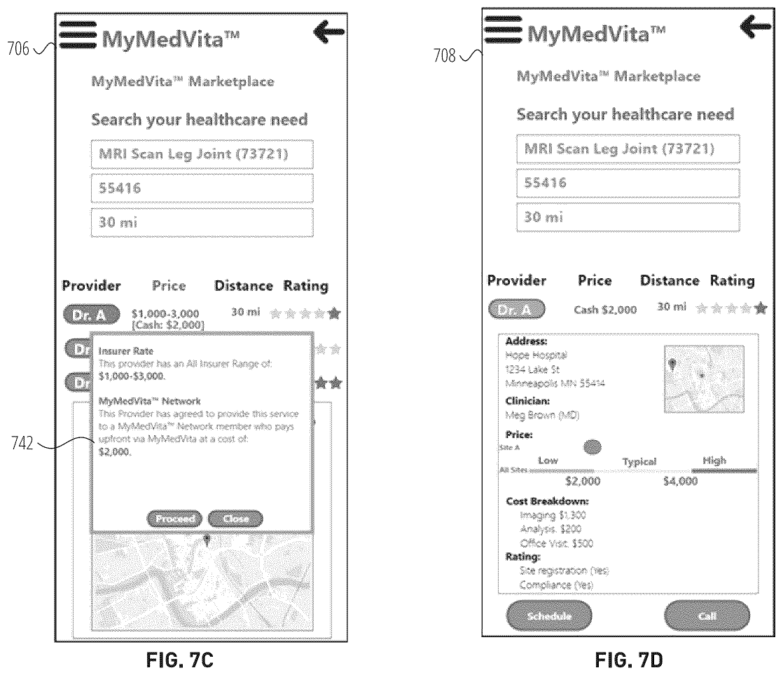

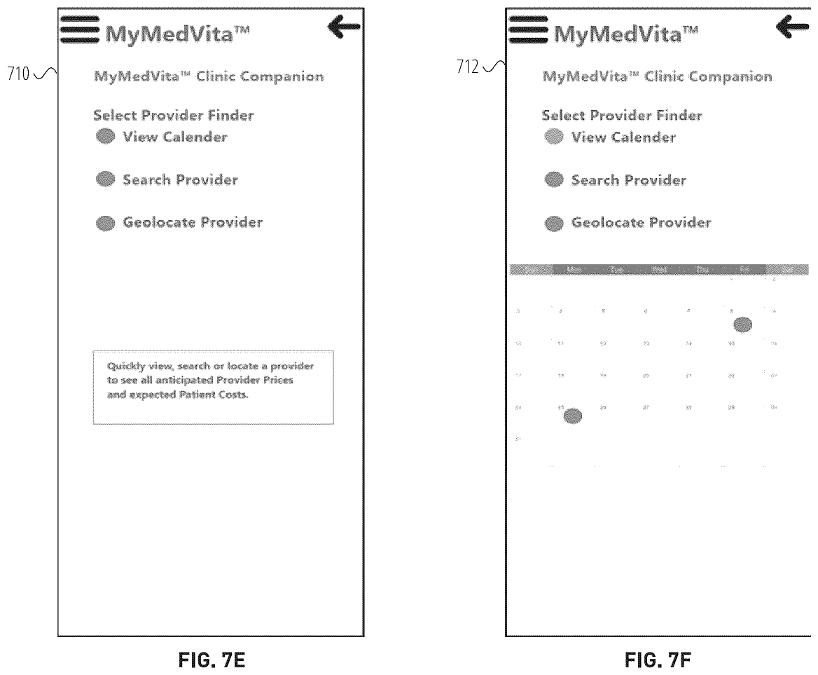

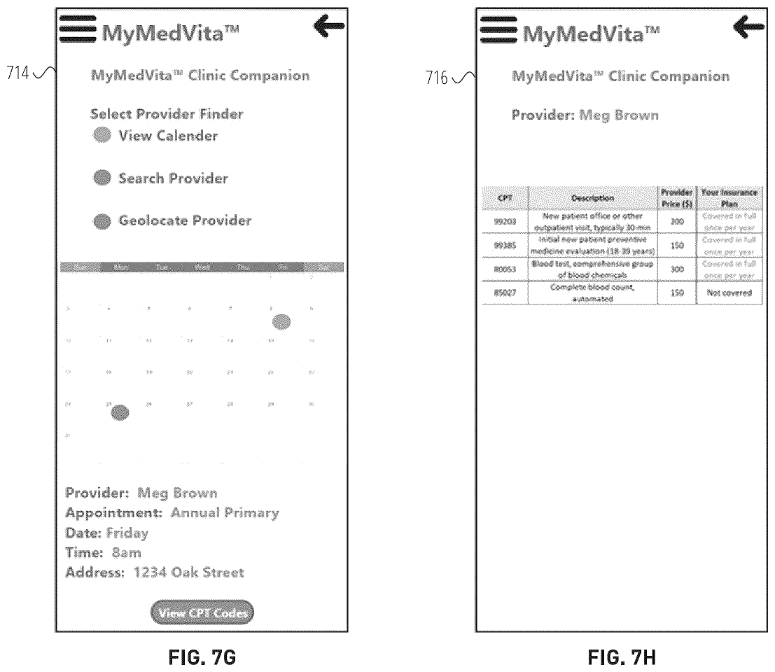

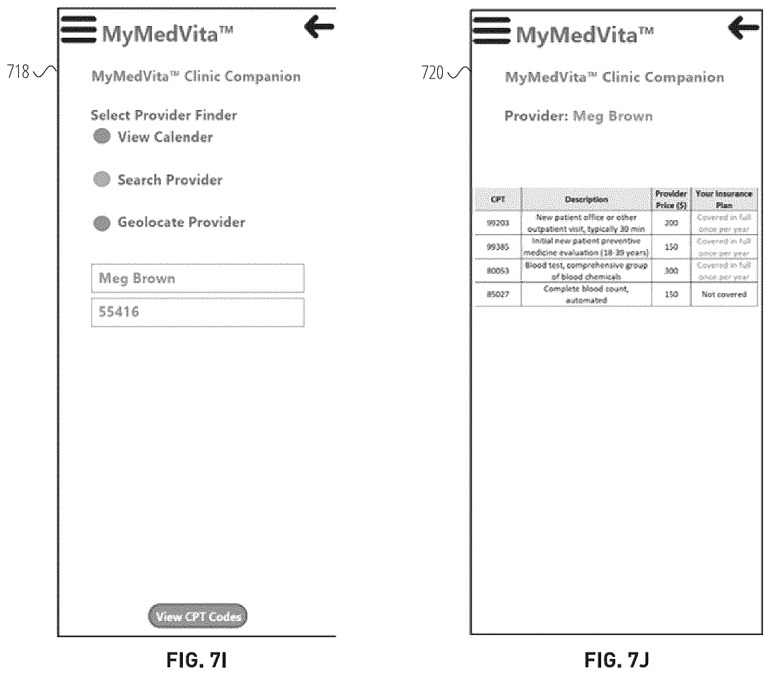

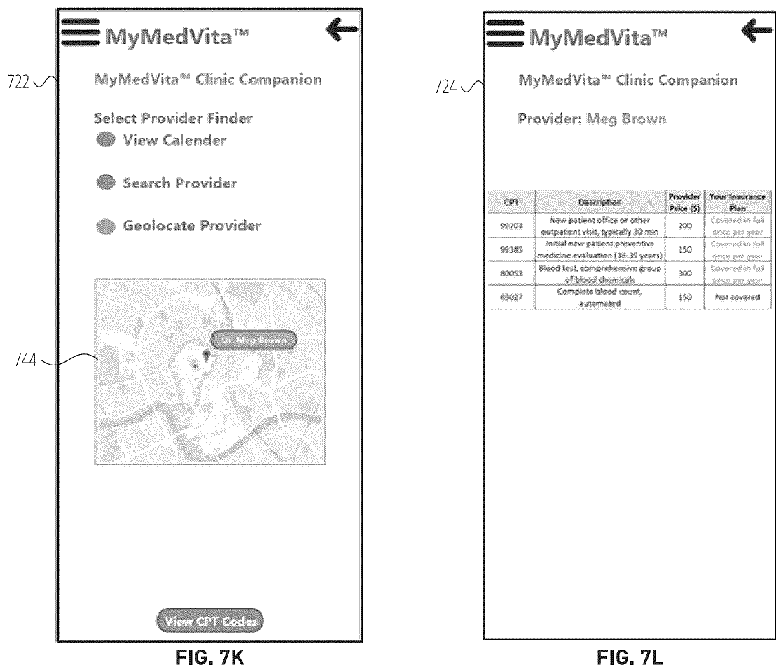

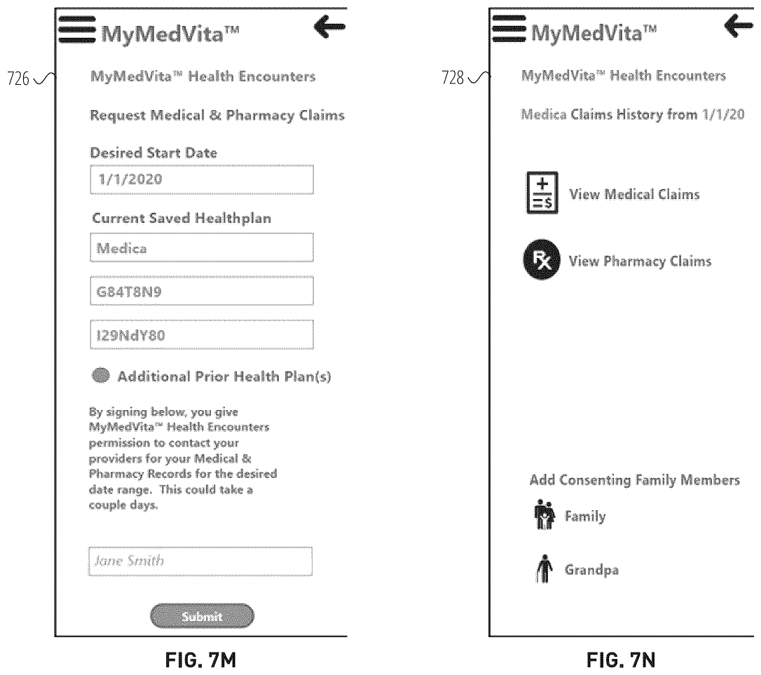

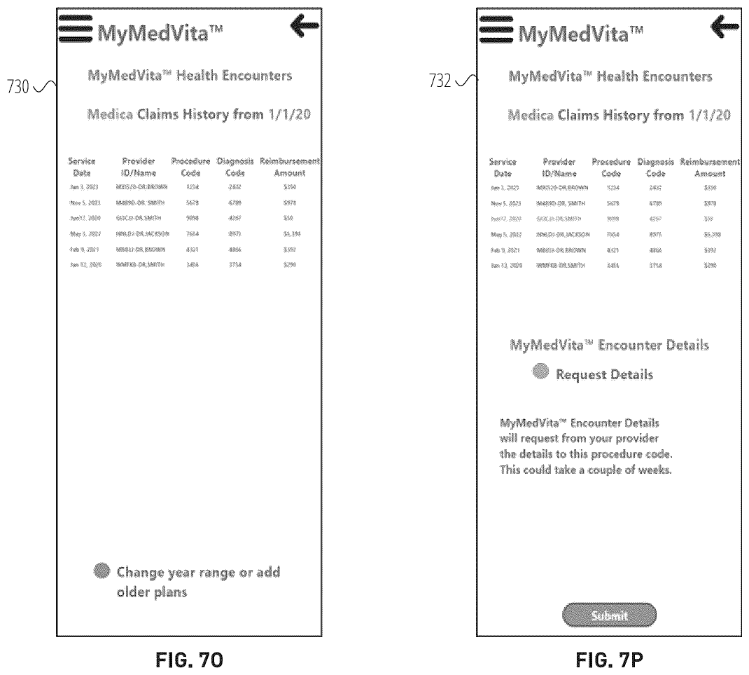

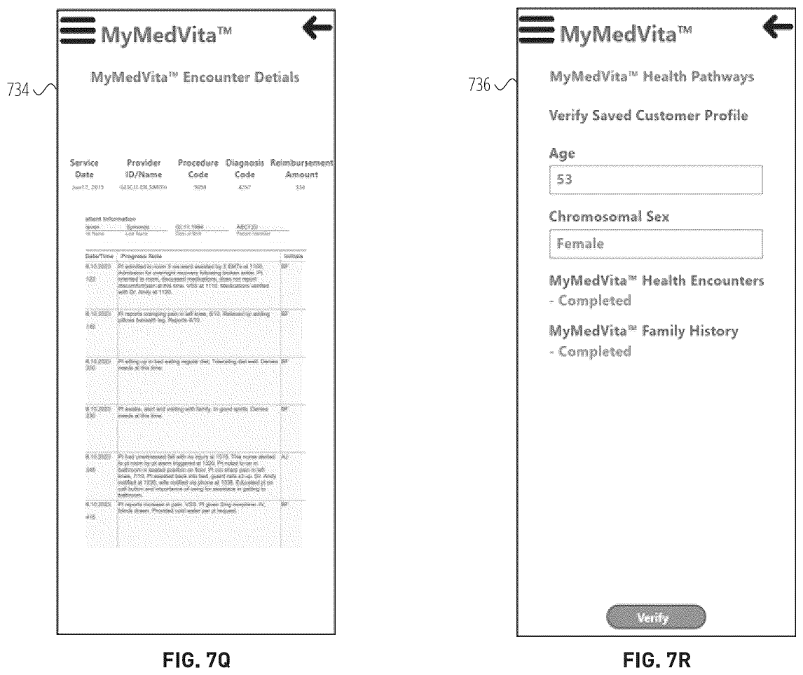

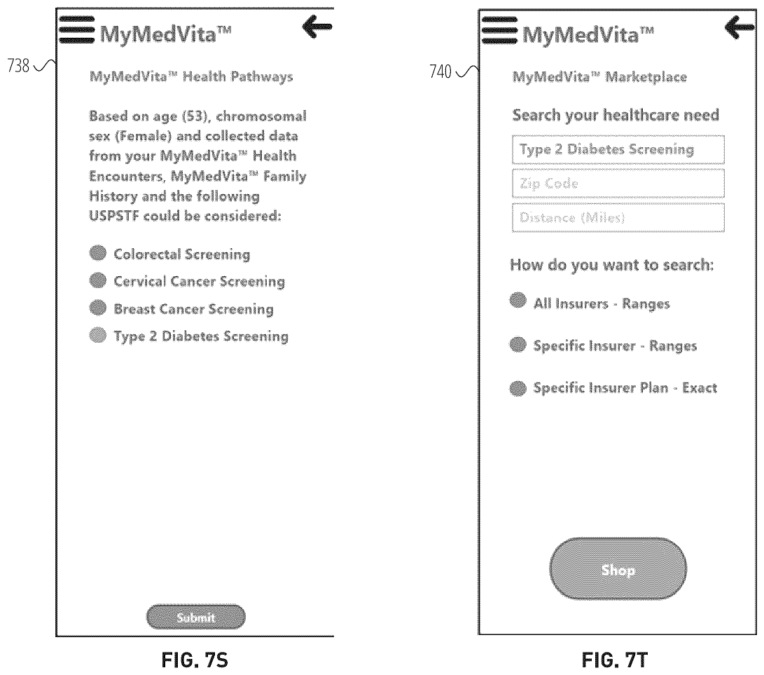

FIGS. 6A to 6S and 7A to 7T illustrate an example embodiment of a user interface for a user device, for example adapted for a mobile device form factor, or alternately adapted for a tablet, laptop, desktop or any other display and interface system.

As illustrated and described, the example mobile app has key benefits including:
Personalized healthcare pricing and cost estimates
Convenient appointment scheduling and payments
Aggregation of medical records from multiple systems
Preventative screening suggestions based on health history
User friendly navigation for healthcare management

In summary, these screenshots illustrate an example end-to-end user experience and key capabilities for shopping, booking, paying, health records access, and preventative screening analytics or other health care that may be performed in the future. The interfaces confer significant improvements in access, convenience and personalization.

As shown in FIG. 6A, there is presented an app home page 602 showing logo, compass graphic, and text stating ‘Your compass for healthcare prices’. Additional text lists abilities to shop, schedule, pay, and review. A ‘Start Shopping’ button is shown.

As shown in FIG. 6B, there is presented a search page 604 for healthcare needs allowing search by MRI scan code. Shows logo, zip code field (or alternatively a field for any other means of identifying a geographic search area), distance slider. ‘How do you want to search’ dropdown with ‘By Insurance Options’ and ‘No Insurance’ options. A ‘Shop’ button is shown.

As shown in FIG. 6C, there is presented a search page 606 with dropdown expanded showing the following insurance search options 640: ‘All Insurers-Ranges’, ‘Specific Insurer-Ranges’, ‘Specific Insurer Plan-Exact’. A ‘Shop’ button is shown.

As shown in FIG. 6D, there is presented a results page 608 showing provider price ranges for all insurers. Data includes provider names, price ranges, distance rating (geographic vicinity or area), low/typical/high price benchmarks. Provider names are links.

As shown in FIG. 6E, there is a user interface 610 with individual provider results expanded to show address, clinician name, rating details, site pricing, and cost breakdown. ‘Schedule’ and ‘Call’ buttons are shown.

As shown in FIG. 6F, there is presented a screenshot 612 of the app's ‘Clinic Companion’ after selecting a provider showing available on-line appointment booking options using an online calendar booking system.

As shown in FIG. 6G, there is a screenshot 614 presented with a selected appointment date and time from Clinic Companion. Shows prompt to log in or create account.

As shown in FIG. 6H, there is a screenshot 616 presented with an account creation form requesting contact information, emergency contact, health insurance information, and payment information. ‘Confirm’ and ‘Submit’ buttons shown.

As shown in FIG. 6I, there is a screenshot 618 presented with a confirmation page after booking appointment. Shows confirmation code and notes details were sent to email and calendar. A ‘Close’ button is shown.

As shown in FIG. 6J, there is a screenshot 620 presented with a search options page for specifying exact insurer plan, with plan details shown.

As shown in FIG. 6K, there is a screenshot 622 presented with a results page filtered to only show ranges negotiated with the specified insurer. Low/typical/high benchmarks still shown.

As shown in FIG. 6L, there is a screenshot 624 presented with an individual provider result expanded showing address, clinician name, rating details, site pricing, and cost breakdown. ‘Schedule’ and ‘Call’ buttons shown.

As shown in FIG. 6M, there is a screenshot 626 presented with a results page shows exact prices negotiated for specified insurer plan. Low/typical/high benchmarks still shown.

As shown in FIG. 6N, there is a screenshot 628 presented with a results page showing provider price ranges for a specific insurer.

As shown in FIG. 6O, there is a screenshot 630 presented with individual provider results expanded to show address, clinician name, rating details, site pricing, and cost breakdown. ‘Schedule’ and ‘Call’ buttons are shown.

As shown in FIG. 6P, there is a screenshot 632 presented with a text-only page 642 explaining provider prices vs. patient costs with insurance specifics entered.

As shown in FIG. 6Q, a user selecting sign-up screenshot 634 is presented with an account creation form requesting contact information, emergency contact, health insurance information, and payment information that can be used to submit to an online payment system. ‘Confirm’ and ‘Submit’ buttons shown.

As shown in FIG. 6R, a results page screenshot 636 is presented showing provider insurer ranges and specific ‘Cash’ prices offered if paying upfront without insurance.

As shown in FIG. 6S, a results page screenshot 638 is presented showing breakdown 644 of cost for a patient.

As shown in FIG. 7A, there is a screenshot 702 presented with individual uninsured search options to obtain provider prices and patient costs based on a medical procedure.

As shown in FIG. 7B, there is a screenshot 704 presented with provider results expanded showing cash price and rating details. Other insurer rates still shown for context.

As shown in FIG. 7C, there is a screenshot 706 presented with a marketplace results page 742 showing provider prices and patient costs for a procedure along with definition of costs.

As shown in FIG. 7D, there is a screenshot 708 presented with a marketplace results page showing provider prices and patient costs for a provider.

As shown in FIG. 7E, there is a screenshot 710 presented with a clinic companion home page showing all search provider options-calendar, search, geolocate.

As shown in FIG. 7F, there is a screenshot 712 presented with a clinic companion calendar view page showing ability to select provider finder options.

As shown in FIG. 7G, there is a screenshot 714 presented with a calendar view showing scheduled provider appointment details.

As shown in FIG. 7H, there is a screenshot 716 presented with a table showing mathematical breakdown of how patient costs are calculated from provider price, deductible etc.

As shown in FIG. 7I, there is a screenshot 718 presented with a search provider results page in clinic companion showing provider selected and option to view CPT codes.

As shown in FIG. 7J, there is a screenshot 720 presented with a table showing mathematical breakdown of how patient costs are calculated from provider price, deductible etc.

As shown in FIG. 7K, there is a screenshot 722 presented with a map 744 view in clinic companion showing pinned provider location. Option to view CPT codes.

As shown in FIG. 7L, there is a screenshot 724 presented with a table showing mathematical breakdown of how patient costs are calculated from provider price, deductible etc.

As shown in FIG. 7M, there is a screenshot 726 presented with a page introducing the app's Health Encounters ability to request medical records. Shows consent signature.

As shown in FIG. 7N, there is a screenshot 728 presented with a page showing medical claims history with table listing claims data. Options to view pharmacy claims or add family members.

As shown in FIG. 7O, there is a screenshot 730 presented with a page showing medical claims history with table listing claims data. Options to view pharmacy claims or add family members.

As shown in FIG. 7P, there is a screenshot 732 presented with an expanded medical claim record showing detailed data. Option to request additional details from provider.

Form showing requested expanded medical claim details were submitted to provider.

As shown in FIG. 7Q there is a screenshot 734 presented with an example individual patient electronic health record showing detailed visit data and progress notes over time.

As shown in FIG. 7R, there is a screenshot 736 presented with a page introducing the app's Health Pathways to suggest screenings based on collected health data.

As shown in FIG. 7S, there is a screenshot 738 presented with a page showing health screening suggestions based on age, sex, and collected health data. Option to search marketplace for the screening.

As shown in FIG. 7T, there is a screenshot 740 presented with a marketplace search page pre-populated for suggested screening. Shows insurance search options.

According to embodiments, there are several types of cloud architectures that could be utilized for the healthcare management system backend including those listed below.

Public Cloud—The system processing components are hosted on shared computing infrastructure from a public cloud provider such as AWS, Azure, or Google Cloud. Resources are available to any public customer with an account.

Private Cloud—The processing components utilize a private cloud environment with dedicated infrastructure only accessible internally behind a firewall. This offers more control but requires owning the hardware.

Hybrid Cloud—Some system components like databases run in a private cloud while other application layers utilize public cloud resources. This offers a balance of control and flexibility.

Multi-Cloud—Different system components could run on different public cloud platforms based on specialized services offered by each. For example, AI/ML on Azure and container apps on AWS.

According to example embodiments, the optimal cloud architecture for a given system will balance factors like cost, flexibility, security and compliance requirements.

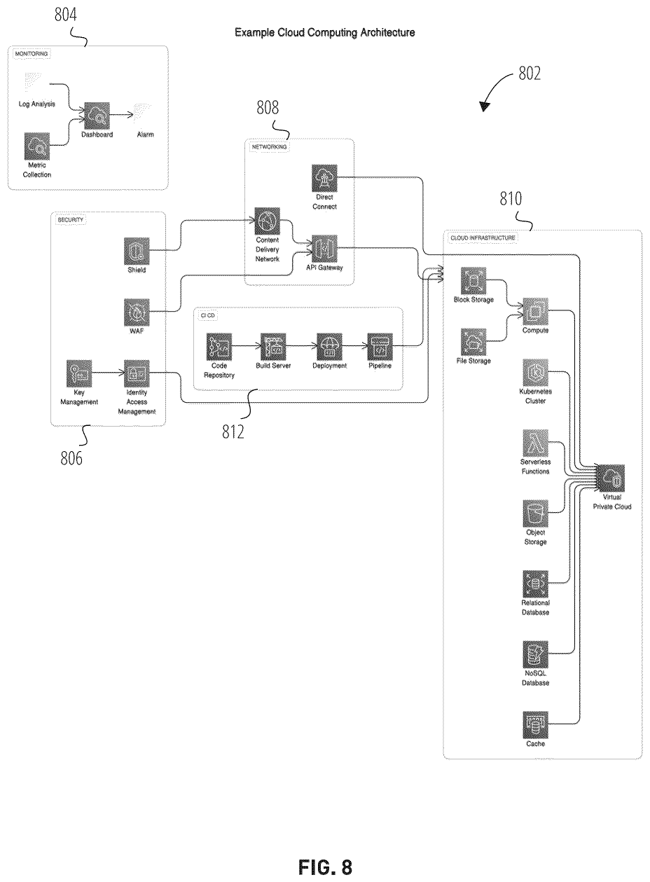

Referring to FIG. 8, there is illustrated one example cloud computing environment 802 that is used to host the server components of an architecture suitable for use to host the capabilities described herein above. As described these capabilities include various processors included in various systems to execute computer program instructions used to execute the various software functions described herein by causing a processor to perform electronic operations. The diagram depicts an example cloud computing architecture. There are several main components shown. In the monitoring section 804, there are blocks labeled Log Analysis, Dashboard, Alarm, Metric Collection, CO, and Co. The security section 806 contains blocks labeled Shield, WAF, Key Management, and Identity Access Management. The networking section 808 shows Direct Connect, Content Delivery Network, API Gateway, and CI CD 812 which connects to a Code Repository, Build Server, and Deployment Pipeline. The cloud infrastructure section 810 contains blocks labeled Block Storage, Compute, File Storage, Kubernetes Cluster, Serverless Functions, Object Storage, Relational Database, NOSQL Database, Cache, and Virtual Private Cloud.

According to one example embodiment, the monitoring section 804 includes:
Log Analysis—Used to aggregate and analyze log data to identify trends, patterns, and anomalies. Helpful for monitoring system health, troubleshooting issues, and security analysis.
Dashboard—Provides visualization and insights into key metrics and data points for monitoring the cloud environment.
Alarm—Can automatically trigger alerts and notifications when certain conditions are met, such as a server going offline or a metric reaching a certain threshold.
Metric Collection—Gathers performance metrics and statistics from resources in the cloud environment like CPU usage, network traffic, etc.
CO—Unclear what this refers to based on the context provided.
Co—Unclear what this refers to based on the context provided.

According to one example embodiment, the security section 806 includes:
Shield—Likely referring to DDOS protection to shield against distributed denial of service attacks.
WAF—Web Application Firewall filters and monitors web traffic to protect against attacks targeting web apps.
Key Management—Manages encryption keys, enabling security measures like encryption and access controls.
Identity Access Management—Manages permissions and access controls for resources and services.

According to one example embodiment, the networking section 808 includes:
Direct Connect—Provides dedicated private connectivity between on-premises network and cloud environment.
Content Delivery Network—Distributes content globally to speed up delivery and improve user experience.
API Gateway—Manages APIs, acts as front door to access backend services.
CI CD—Continuous integration and continuous delivery automates building, testing and deployment of applications. Connects to:
   Code Repository—Stores source code.
   Build Server—Automates compiling source code into executable applications.
   Deployment Pipeline—Automates release process including building, testing and deploying new versions.

According to one example embodiment, the cloud infrastructure section 810 includes:
Block Storage—Persistent block-level storage volumes for EC2 instances.
Compute—Refers to virtual server compute capacity provided by EC2 virtual machines.
File Storage—Shared file storage like Elastic File System (EFS).
Kubernetes Cluster—Groups and manages docker containers and scheduling based on resource utilization.
Serverless Functions—Enables running code in response to events without managing servers.
Object Storage—Scalable storage for data objects via services like S3.
Relational Database—Managed relational databases like AWS RDS.
NoSQL Database—Managed non-relational distributed databases.
Cache—In-memory caching improves performance by storing frequently accessed data.
Virtual Private Cloud—Logically isolated virtual network to run resources in virtual data centers.
   The various storage devices and systems noted above provide various non-transitory computer-readable storage mediums used to store computer program instructions used to execute the various software functions described herein.

According to other example embodiments client computers include various processors included in various processing systems to execute computer program instructions to cause a processor to perform electronic operations and include:
Personal computers (desktop PCs, laptops)
Mobile devices (smartphones, tablets)
Thin clients
Workstations
Gaming consoles
Smart TVs
Internet of things (IOT) devices
Virtual reality headsets

The client computing systems would access, interact with, and leverage services provided by servers in a client-server architecture. The servers would handle most of the centralized data storage and processing, while the client devices would provide the user interface and local processing power.

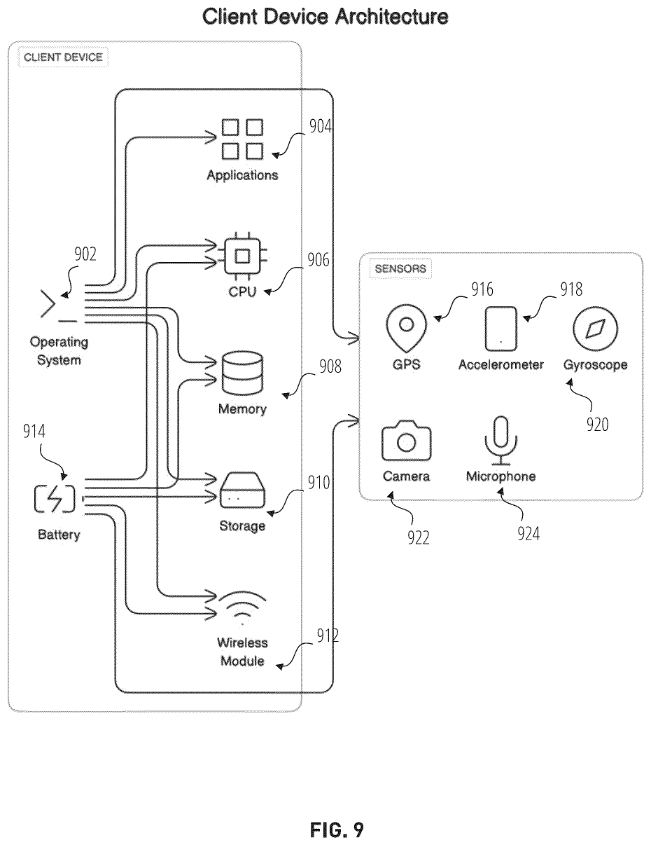

One example embodiment of a client computer is illustrated in FIG. 9 and includes the following components:
Operating System 902—The core software on a device that manages hardware and software resources and provides common services for applications. It manages memory, processes, file systems, networking, etc.
Battery 914—Provides power to operate the device. Lithium-ion batteries are common. Battery life is a key factor for mobile devices.
Client Device Architecture—The core hardware components and how they are organized. This includes the CPU, memory, storage, input/output modules, etc.
Applications 904—Software programs running on the device that perform specific functions for the user. Email, web browsers, games, and productivity apps are common.
CPU 906—The central processing unit that executes instructions and performs calculations. Common architectures are ARM and x86. More cores and higher clock speeds equate to faster processing.
GPS 916—Global Positioning System receiver that calculates location using satellite signals. Enables location-aware features and mapping.
Accelerometer 918—Measures acceleration forces along reference axes. Used for motion detection in games, screen rotation, etc.
Gyroscope 920—Measures orientation and rotation. Used with accelerometer for advanced motion sensing.
Memory 908—Temporary storage used by running programs. More RAM allows more applications to run. Common types are LPDDR4 and LPDDR5.
Camera 922—Image sensor that captures photographs and video. Key specs are megapixels, aperture, zoom, and low light performance.
Microphone 924.
Audio sensor that records sound. Used for video, voice calls, voice assistants, etc.
Storage 910
Permanent storage for the operating system, applications, photos, media, and other user files. Flash or solid state is common.
Wireless Module 912
Hardware for connecting to wireless networks like WiFi and mobile data. Supports communications and internet connectivity.

While initially designed for the U.S. healthcare system, the healthcare management system is architected with international expansion in mind. The system's modular design allows for easy adaptation to different healthcare systems, regulations, and languages. Region-specific modules can be developed to handle unique aspects of various national healthcare systems, such as different insurance models or government health programs. This flexibility positions the system to potentially revolutionize healthcare management on a global scale, improving access and efficiency in diverse healthcare environments worldwide.

Thus, as described above there is provided a system comprising various interfaces and an analytics engine to assist users in finding and booking healthcare providers, viewing negotiated prices, accessing health records, making payments, and receiving preventative care recommendations based on their health data.

The healthcare management system is designed with a strong commitment to accessibility, ensuring that users of all abilities can effectively utilize its features. The user interface is fully compatible with screen readers and supports voice commands for hands-free operation. Text size and contrast can be easily adjusted to accommodate visual impairments. The system also includes features such as closed captioning for video content and alternative text for images. These accessibility features not only comply with ADA standards but also reflect the system's commitment to inclusive healthcare management.

The system includes a search interface to find providers based on search criteria, a results interface displaying insurer-negotiated prices, a provider rating display interface, a scheduling interface for booking appointments, a payment interface for directly paying providers, and a health records interface for viewing user health records and claims data.

The system also includes an analytics engine that utilizes algorithms to suggest future preventative healthcare based on the user's health records and claims data. This can involve comparing the user's health data against clinical guidelines to recommend preventative screening tests or identify potential medical conditions.

Additional features include a geolocation interface to find providers within a specified geographic area, the ability to book appointments directly through an online calendar system, connecting to the user's payment information for direct online payments, and gathering health records and claims data from various electronic systems and insurance providers.

The search criteria can include a medical procedure billing code to find providers performing a specific medical procedure desired by the user.

The system, method, and non-transitory computer-readable storage medium claims describe various implementations and embodiments of this comprehensive healthcare provider search, booking, payment, records access, and preventative care recommendation system.

Thus, according to one embodiment of the system, article of manufactures and methods described herein, there is provided a system for assisting users in finding and utilizing healthcare providers and services. The system comprises a search interface that allows users to find providers providing a service based on specified search criteria. It also includes a results interface that displays insurer-negotiated prices for the providers of the desired service. The system further comprises a provider rating display interface for viewing ratings or reviews of providers. To facilitate scheduling appointments, the system provides a scheduling interface that enables users to book appointments directly with providers. Additionally, there is a payment interface that allows users to directly pay providers, likely connecting to the user's payment information for online payments. The system also incorporates a health records interface where users can view their own health records and insurance claims data. This health data is utilized by an analytics engine within the system. The analytics engine employs algorithms to analyze the user's health records and claims data, suggesting future preventative healthcare measures based on this analysis. In another embodiment, the system further comprises a geolocation interface to find providers within a geographic search area centered on a user-specified location, allowing users to search for nearby providers. In another embodiment, the scheduling interface specifically allows users to book appointments directly with providers through an online calendar booking system for convenient scheduling. In another embodiment, the payment interface connects to the user's payment information, such as a credit card on file, enabling direct payments to providers through an online payment system integrated into the system. In another embodiment, the health records interface gathers the user's health records and insurance claims data from various external electronic health record systems and insurance providers to consolidate the information within the system. In another embodiment, the analytics engine compares the user's health data against clinical guidelines to provide tailored suggestions, such as recommending preventative health screening tests or identifying potential medical conditions the user may be at risk for. In another embodiment, the search criteria used to find providers can comprise a specific medical procedure billing code, allowing users to search for providers that perform a particular medical procedure the user needs. The above embodiments can be implemented as a system, as a method with the various components being provided, or as a non-transitory computer-readable storage medium storing instructions for a processor to execute the method.

Thus, as described herein there is provided a user interface system providing a comprehensive platform for managing healthcare services and obtaining value-driven services based on various factors and comparative shopping. It allows users to search for healthcare providers based on their specific health needs or medical conditions, as well as the corresponding medical procedure billing codes.

One embodiment of the system includes a search interface that enables users to find healthcare providers based on specified search criteria, such as their health need or medical condition, and the associated medical procedure billing code. The results interface displays insurer-negotiated prices for the healthcare providers that offer the medical procedure corresponding to the billing code. Additionally, a provider rating interface displays ratings or reviews for the healthcare providers.

The system also includes a scheduling interface that allows users to directly book appointments with selected healthcare providers through an online calendar booking system. A payment interface enables users to directly pay healthcare providers through an online payment system by connecting to their payment information.

Furthermore, the system features a health records interface that allows users to view their personal health records and insurance claims data gathered from electronic health record systems and insurance providers.

In another embodiment, the system includes a geolocation interface that allows users to find healthcare providers within a geographic search area centered on a user-specified location. The search criteria can also include a geographic location and a distance radius from that location for finding healthcare providers.

The results interface can be configured to display provider pricing information categorized by insurance plan options, such as displaying pricing for all insurers, a specific insurer, or a specific insurer plan.

The provider rating interface may display provider ratings based on user reviews.

An account creation interface can be included to allow users to create an account by providing contact information, emergency contact information, health insurance information, and payment information.

The scheduling interface can display a calendar view of available appointment times for a selected healthcare provider.

The payment interface can calculate patient costs for a healthcare service based on the insurer-negotiated pricing and the user's health insurance information.

The health records interface can display medical claims data and pharmacy claims data of the user, and may also allow users to request additional details related to medical claims from healthcare providers. It can display consolidated health records for the user gathered from multiple healthcare providers.

In another embodiment, the system includes a family member interface that allows users to view and manage health records for family members.

The search interface can be configured to allow users to search for healthcare providers offering specific medical screenings or preventative services.

A provider map interface can display geographic locations of healthcare providers on an interactive map.

A cost breakdown interface can display a mathematical breakdown of how patient costs are calculated from provider pricing, insurance deductibles, and other factors.

The search interface can allow users to search for healthcare providers without providing insurance information to obtain uninsured cash pricing.

A consent interface can be included to allow users to provide consent for accessing their personal health records and insurance claims data.

The system may also include a family history interface that allows users to input family medical history information, and a screening recommendations interface that displays suggested preventative health screening procedures for the user based on their age, biological sex, and health records.

According to one embodiment, Artificial Intelligence (AI) and Machine Learning (ML) are core components of the healthcare management system, enhancing its capabilities and user experience. AI algorithms analyze vast amounts of healthcare data to improve price predictions, offering users more accurate cost estimates for medical services. ML models personalize health recommendations based on individual user profiles, health histories, and behaviors. The system's AI also continuously optimizes the user interface, adapting to user preferences and patterns to provide a more intuitive and efficient experience.

While various embodiments of methods and systems have been described above, it should be understood that they have been presented by way of example only, and not limitation. Thus, the breadth and scope of the technology described herein should not be limited by any of the above-described exemplary embodiments, but should be defined only in accordance with the following claims and their equivalents.

What is claimed is:

1. A user interface system for managing healthcare services, comprising:
a search interface configured to allow a user to find healthcare providers based on specified search criteria, wherein the search criteria includes a health need or medical condition of the user that corresponds to a billing code for a medical procedure;
a results interface configured to display insurer-negotiated pricing for the healthcare providers that offer the medical procedure corresponding to the billing code;
a provider rating interface configured to display ratings or reviews for the healthcare providers;
a scheduling interface configured to allow the user to directly book appointments with selected healthcare providers through an online calendar booking system;
a payment interface configured to allow the user to directly pay healthcare providers through an online payment system by connecting to payment information of the user; and
a health records interface configured to allow the user to view their personal health records and insurance claims data gathered from electronic health record systems and insurance providers.

2. The user interface system of claim 1, further comprising a geolocation interface configured to allow the user to find healthcare providers within a geographic search area centered on a user-specified location.

3. The user interface system of claim 1, wherein the search criteria further includes a geographic location and a distance radius from the geographic location for finding healthcare providers.

4. The user interface system of claim 1, wherein the results interface is further configured to display provider pricing information categorized by insurance plan options.

5. The user interface system of claim 4, wherein the insurance plan options include displaying pricing for all insurers, a specific insurer, or a specific insurer plan.

6. The user interface system of claim 1, wherein the provider rating interface displays provider ratings based on user reviews.

7. The user interface system of claim 1, further comprising an account creation interface configured to allow the user to create an account by providing contact information, emergency contact information, health insurance information, and payment information.

8. The user interface system of claim 1, wherein the scheduling interface is configured to display a calendar view of available appointment times for a selected healthcare provider.

9. The user interface system of claim 1, wherein the payment interface is configured to calculate patient costs for a healthcare service based on the insurer-negotiated pricing and health insurance information for the user.

10. The user interface system of claim 1, wherein the health records interface is configured to display medical claims data and pharmacy claims data of the user.

11. The user interface system of claim 10, wherein the health records interface is further configured to allow the user to request additional details related to medical claims from healthcare providers.

12. The user interface system of claim 1, wherein the health records interface is configured to display consolidated health records for the user gathered from multiple healthcare providers.

13. The user interface system of claim 1, further comprising a family member interface configured to allow the user to view and manage health records for family members.

14. The user interface system of claim 1, wherein the search interface is further configured to allow the user to search for healthcare providers offering a specific medical screening or preventative service.

15. The user interface system of claim 1, further comprising a provider map interface configured to display geographic locations of healthcare providers on an interactive map.

16. The user interface system of claim 1, further comprising a cost breakdown interface configured to display a mathematical breakdown of how patient costs are calculated from provider pricing, insurance deductibles, and other factors.

17. The user interface system of claim 1, wherein the search interface is further configured to allow the user to search for healthcare providers without providing insurance information to obtain uninsured cash pricing.

18. The user interface system of claim 1, further comprising a consent interface configured to allow the user to provide consent for accessing the personal health records and insurance claims data of the user.

19. The user interface system of claim 1, further comprising a screening recommendations interface configured to display suggested preventative health screening procedures for the user based on the age, biological sex, and health records of the user.

20. A user interface system of claim 1 further including an analytics engine configured to: receive the personal health records and insurance claims data of the user from the health records interface; analyze the personal health records and insurance claims data using machine learning models trained on population health data; identify preventative health screening procedures relevant to the user based on the analysis; and a screening recommendations interface configured to display the identified preventative health screening procedures along with the ability to use the search interface to find healthcare providers offering the identified screening procedures.
